(19) United States
(12) Patent Application Publication
(10) Pub. No.: US 2013/0317297 A1
(43) Pub. Date: Nov. 28, 2013

(54) CAPSULE ENDOSCOPE

(71) Applicant: Chung-che WU, Taipei City (TW)

(72) Inventors: Chung-che WU, Taipei City (TW); Kai-yun Chen, Taipei City (TW)

(21) Appl. No.: 13/899,605

(22) Filed: May 22, 2013

Related U.S. Application Data
(60) Provisional application No. 61/649,944, filed on May 22, 2012.

(51) Int. Cl. A61B 1/04; A61B 1/00

(52) U.S. Cl. CPC A61B 1/041; A61B 1/00181; A61B 1/00009; A61B 1/0002; A61B 1/00016; A61B 1/00147
USPC 600/109

(57) ABSTRACT

A capsule endoscope is disclosed. The capsule endoscope includes a shell, a plurality of image capturing units, a positioning unit, and a processing unit. The shell has a containing space. The image capturing units are disposed in the containing space and arranged such that the image capturing units capture images from different angles. The positioning unit is disposed in the containing space for positioning each of the image capturing units. The processing units is disposed in the containing space for acquiring angles of capturing images with the image capturing units according to positioning results of the image capturing units with the positioning unit.

### CROSS REFERENCE TO RELATED APPLICATIONS

This application claims the benefit of the filing date of U.S. provisional patent application Ser. No. 61/649,944, filed on May 22, 2012, the disclosure of which is incorporated herein by reference.

### BACKGROUND OF THE INVENTION

1. Field of the Invention

The present invention generally relates to an endoscope, and more particularly to a capsule endoscope.

2. Description of Prior Art

A traditional endoscope comprises a tube and a camera which is disposed at a distal end of the tube. During examining processes, the tube is inserted to a location to be inspected, for example, an intestine or a stomach. However, this type of traditional endoscope makes a patient uncomfortable. Furthermore, some positions cannot be inspected during the examining processes.

Accordingly, a type of capsule endoscope is developed recently. In the capsule endoscope, a camera is disposed in a capsule. During the examining processes, the patient swallows the capsule endoscope firstly. The capsule endoscope passes through esophagus of the patient and advances to an abdominal cavity due to peristalsis of intestines. Then, the camera of the capsule endoscope captures images. The captured images are served as bases of inspecting the patient.

Since only one camera is disposed in the capsule endoscope, images from a part of angles can be captured but images from another part of angles cannot be captured during the advancing processes. Furthermore, the capsule endoscope advances by a rolling manner. Accordingly, an angle and a capturing range of the camera cannot be controlled, and some of the captured images fail to meet requirements, such that results of inspecting the patient are not accurate.

Furthermore, an image of one location is captured from one angle but not from several different angles. As a result, it fails to utilize images from the several different view angles to reconstruct a three-dimensional (3D) image. That is, only a two-dimensional image can be acquired, and an integral and effective judgment cannot be made.

Consequently, there is a need to solve the above-mentioned problems that the images from some angles cannot be captured and the three-dimensional image cannot be reconstructed in the conventional capsule endoscope.

### SUMMARY OF THE INVENTION

An objective of the present invention is to provide a capsule endoscope which is capable of solving the problems that images from some angles cannot be captured and a three-dimensional image cannot be reconstructed in the conventional capsule endoscope.

To achieve the above-mentioned objective, a capsule endoscope is provided according to an aspect of the present invention. The capsule endoscope comprises a shell, a plurality of image capturing units, a positioning unit, and a processing unit. The shell has a containing space. The image capturing units are disposed in the containing space and arranged such that the image capturing units capture images from different angles. The positioning unit is disposed in the containing space for positioning each of the image capturing units. The processing unit is disposed in the containing space for acquiring angles of capturing images with the image capturing units according to positioning results of the image capturing units with the positioning unit.

To achieve the above-mentioned objective, a capsule endoscope is provided according to another aspect of the present invention. The capsule endoscope comprises a shell, a plurality of image capturing units, a positioning unit, and a processing unit. The shell has a containing space. The image capturing units are disposed in the containing space and arranged such that a capturing range of each of the image capturing units overlaps with a capturing range of at least one adjacent image capture unit. The positioning unit is disposed in the containing space for positioning each of the image capturing units. The processing unit is disposed in the containing space for acquiring angles of capturing images with the image capturing units according to positioning results of the image capturing units with the positioning unit.

The capsule endoscope of the present invention is capable of capturing the images from different angles. That is, all of the image capturing units can capture the images from 360 degrees. Furthermore, since one location is captured by the image capturing units from different angles, the 3-dimensional image can be reconstructed by utilizing the images from different angles for making an integral and effective judgment.

### BRIEF DESCRIPTION OF THE DRAWINGS

FIG. 1 shows an appearance of a capsule endoscope according to a first embodiment of the present invention;

FIG. 2 shows a block diagram of the capsule endoscope in FIG. 1;

FIG. 3 is an example showing that the capturing range of each image capturing unit is different;

FIG. 4 shows a method of sensing the magnetic vector;

FIG. 5 shows a block diagram of the capsule endoscope according to a second embodiment of the present invention;

FIG. 6 shows a block diagram of the capsule endoscope according to a third embodiment of the present invention; and

FIG. 7 shows a block diagram of the capsule endoscope according to a fourth embodiment of the present invention and a receiving unit.

### DETAILED DESCRIPTION OF THE INVENTION

For a better understanding of the aforementioned content of the present invention, preferable embodiments are illustrated in accordance with the attached figures for further explanation

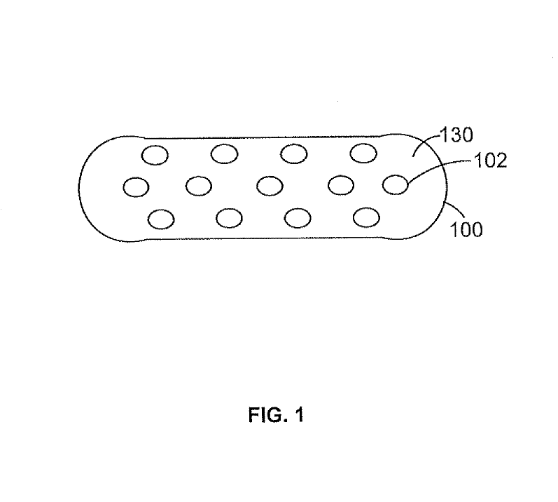

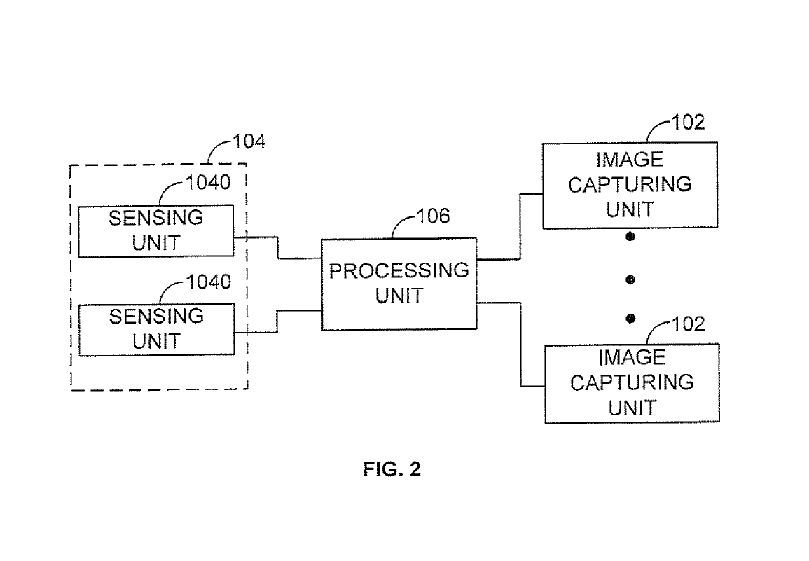

Please refer to FIG. 1 and FIG. 2. FIG. 1 shows an appearance of a capsule endoscope according to a first embodiment of the present invention. FIG. 2 shows a block diagram of the capsule endoscope in FIG. 1.

The capsule endoscope of the present invention comprises a shell 100, a plurality of image capturing units 102, a positioning unit 104, and a processing unit 106.

In the present embodiment, the shell 100 is elliptical. In another embodiment, the shell 100 may be another shape, for instance, circular. The shell 100 has a containing space 130. The image capturing units 102, the positioning unit 104, and the processing unit 106 are disposed in the containing space 130. The shell 100 is a transparent shell. The image capturing units 102 is capable of capturing images through the transparent shell.

It is noted that FIG. 1 shows that the image capturing units 102 are disposed in the containing space 130. The other elements comprising the positioning unit 104 and the processing unit 106 may be disposed in suitable locations of the containing space 130 and are not repeated herein.

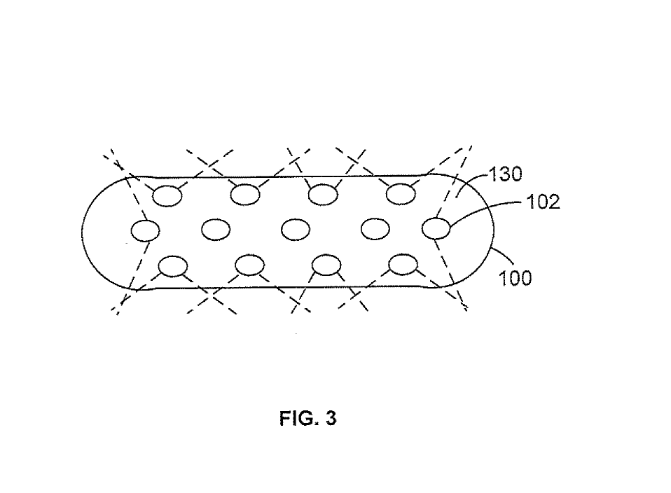

One feature of the present invention is that the capsule endoscope comprises the plurality of image capturing units 102. The image capturing units 102 are arranged such that the image capturing units 102 are capable of capturing images from all different (various) angles. That is, an arrangement of the image capturing units 102 makes the image capturing units 102 capture the images from 360 degrees when the capsule endoscope is static or rolling. It is noted that the arrangement of the image capturing units 102 is not limited. Each image capturing unit 102 may be equidistant or nonequidistant from each other. A capturing range of each image capturing unit 102 may be the same as or different from each other. FIG. 3 is an example showing that the capturing range of each image capturing unit 102 is different. For instance, some image capturing units 102 have a capturing range of 70 degrees, while others have a capturing range of 30 degrees. The image capturing units 102 are arranged such that an image from each angel can be captured by at least one of the image capturing units 102. That is, an objective of capturing the images from 360 degrees can be achieved.

It can be known from FIG. 3 that the image capturing units 102 are disposed in the containing space 130 and arranged such that the capturing range of one image capturing unit 102 overlaps with the capturing range of at least one adjacent image capturing unit 102. As a result, the objective of capturing the images from 360 degrees can be achieved.

it is noted that an amount of the image capturing units 102 may be varied according to requirements. For example, when the capturing range of each image capturing unit 102 is small, the required amount of the image capturing units 102 is high. When the capturing range of each image capturing unit 102 is large, the required amount of the image capturing units 102 is low.

Please refer to FIG. 2. The positioning unit 104 is utilized for positioning each of the image capturing units 102. More particularly, after a patient swallows the capsule endoscope of the present invention, the positioning unit 104 acquires angles of capturing the images with the image capturing unit 102. For instance, each image capturing unit 102 may be positioned by sensing two vectors. In the present embodiment, the positioning unit 104 comprises two sensing units 1040. One of the sensing units 1040 is utilized for sensing a gravity vector, and the other one of the sensing units 1040 is utilized for sensing a magnetic vector.

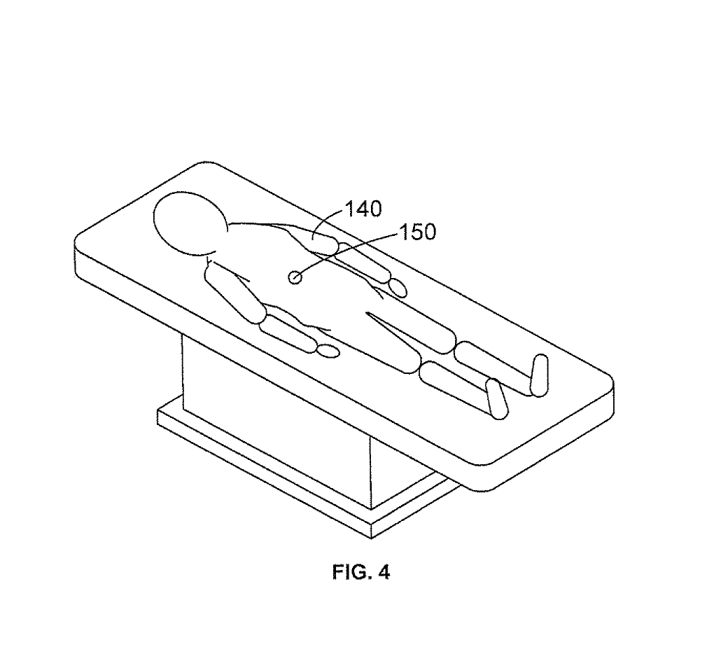

Please refer to FIG. 4. FIG. 4 shows a method of sensing the magnetic vector. In the method, a magnet 150 is disposed in an external location of a patient 140, for instance, a belly. The magnetic vector may be acquired by sensing the location of the magnet 150 with one of the sensing units 1040 in FIG. 2. It is noted that the location of the magnet 150 is not limited to the belly of the patient 140. The position of the magnet 150 may be disposed in any positions where the sensing unit 1040 can sense the magnetic force of the magnet 150.

After the two sensing units 1040 respectively sense the gravity vector and the magnetic vector. Information about the gravity vector and the magnetic vector is sent to the processing unit 106. The processing unit 106 acquires the angles of capturing the images with the image capturing units 102 according to the gravity vector and the magnetic vector. As a result, an objective of positioning each of the image capturing units 102 may be achieved.

In the present embodiment, the processing unit 106 has a built-in timer (not shown). The processing unit 106 may further acquire time of capturing the images according to time of the timer when the image capturing units 102 capture the images.

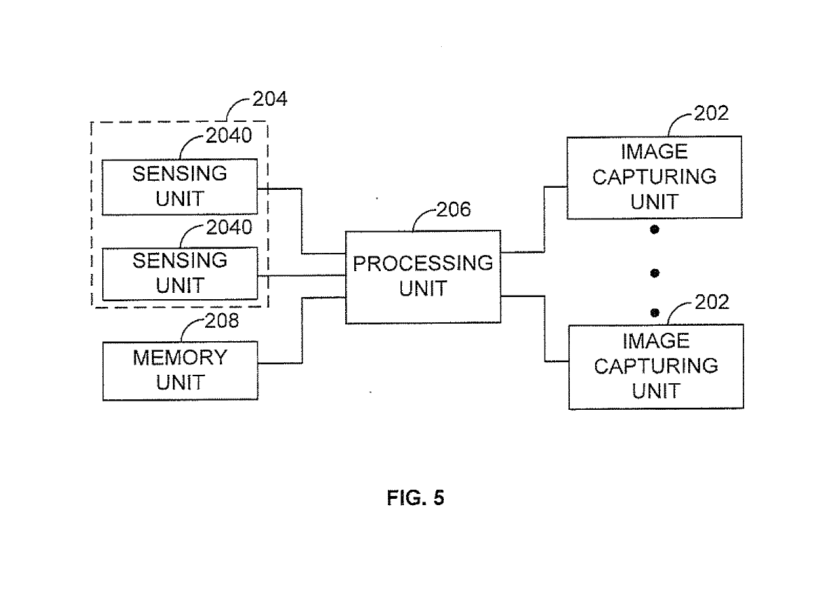

Please refer to FIG. 5. FIG. 5 shows a block diagram of the capsule endoscope according to a second embodiment of the present invention.

In the second embodiment, the capsule endoscope comprises the shell 100 in FIG. 1, a plurality of image capturing units 202, a positioning unit 204, a processing unit 206, and a memory unit 208.

The image capturing units 202, the positioning unit 204, and the processing unit 206 are respectively the same as the image capturing units 102, the positioning unit 104, and the processing unit 106 in the first embodiment and not repeated herein. A difference between the first embodiment and the present embodiment is that the capsule endoscope of the present embodiment further comprises the memory unit 208 for storing images captured by the image capturing units 202 and angles of capturing the images with the image capturing units 202.

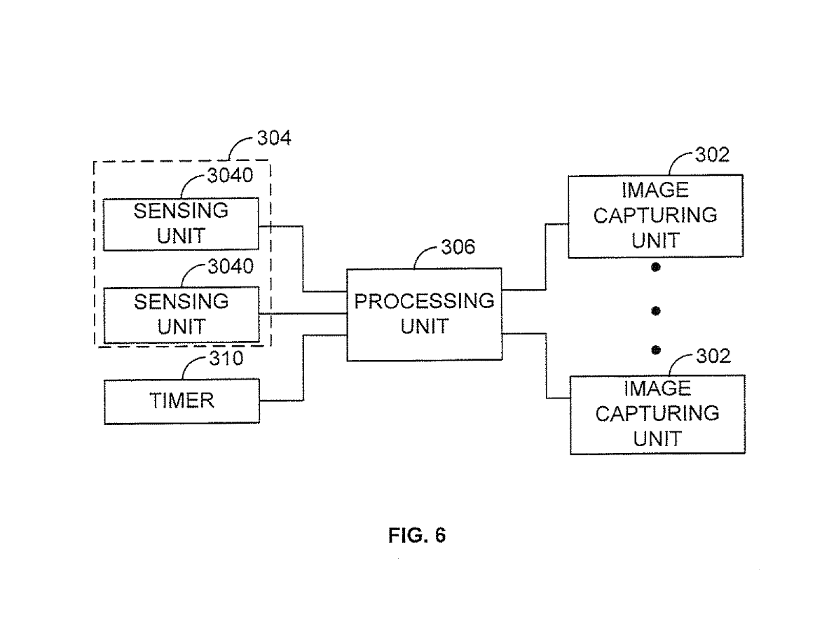

Please refer to FIG. 6. FIG. 6 shows a block diagram of the capsule endoscope according to a third embodiment of the present invention.

In the third embodiment, the capsule endoscope comprises the shell 100 in FIG. 1, a plurality of image capturing units 302, a positioning unit 304, a processing unit 306, and a timer 310.

The image capturing units 302, the positioning unit 304, and the processing unit 306 are respectively the same as the image capturing units 102, the positioning unit 104, and the processing unit 106 in the first embodiment and not repeated herein. A difference between the first embodiment and the present embodiment is that the timer 310 is independently disposed for recording time in the present embodiment. The timer (now shown) in the first embodiment is built in the processing unit 106. The processing unit 106 may determine a capturing sequence of all images according to angles of capturing the images with the image capturing units 302 and the time of the timer 310. Thereafter, a three-dimensional image may be reconstructed by utilizing some of the images according to requirements.

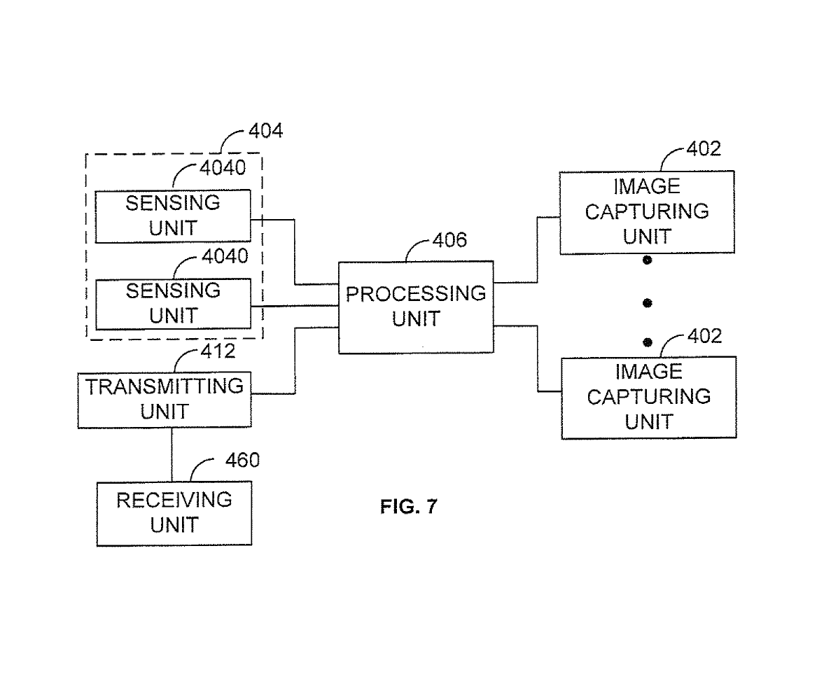

Please refer to FIG. 7. FIG. 7 shows a block diagram of the capsule endoscope according to a fourth embodiment of the present invention and a receiving unit.

In the fourth embodiment, the capsule endoscope comprises the shell 100 in FIG. 1, a plurality of image capturing units 402, a positioning unit 404, a processing unit 406, and a transmitting unit 412.

The image capturing units 402, the positioning unit 404, and the processing unit 406 are respectively the same as the image capturing units 102, the positioning unit 104, and the processing unit 106 in the first embodiment and not repeated herein. A difference between the first embodiment and the present embodiment is that the capsule endoscope of the present embodiment further comprises the transmitting unit 412 for transmitting captured images and angles of capturing the images with the image capturing units 402 to a receiving unit 460 in an external location of a patient. An image processing system (not shown) processes the captured images and the angles of capturing the images with the image capturing units 402.

It is noted that the capsule endoscope in FIG. 1 may comprise at least one of the memory unit 208 in FIG. 5, the timer 310 in FIG. 6, and the transmitting unit 460 in FIG. 7 according to requirements.

The capsule endoscope of the present invention comprises a plurality of image capturing units, and the image capturing units are arranged such that the image capturing units capture images from all angles. That is, all of the image capturing units can capture the images from 360 degrees. Furthermore, the angles of capturing the images may be acquired by positioning each of the image capturing units, and the time of capturing the images may be acquired with the timer. Accordingly, the required images may be found easily for increasing judgment accuracy. Finally, since one location is captured by the image capturing units from different angles, the 3-dimensional image can be reconstructed by utilizing the images from different angles for making an integral and effective judgment.

As is understood by a person skilled in the art, the foregoing preferred embodiments of the present invention are illustrative rather than limiting of the present invention. It is intended that they cover various modifications and similar arrangements be included within the spirit and scope of the appended claims, the scope of which should be accorded the broadest interpretation so as to encompass all such modifications and similar structure.

What is claimed is:

1. A capsule endoscope, comprising:
a shell having a containing space;
a plurality of image capturing units disposed in the containing space and arranged such that the image capturing units capture images from different angles;
a positioning unit disposed in the containing space for positioning each of the image capturing units: and
a processing unit disposed in the containing space for acquiring angles of capturing images with the image capturing units according to positioning results of the image capturing units with the positioning unit.

2. The capsule endoscope of claim 1, wherein the positioning unit positions each of the image capturing units by utilizing two vectors.

3. The capsule endoscope of claim 2, wherein the positioning unit comprises two sensing units, one of the sensing units is utilized for sensing a gravity vector, and the other one of the sensing units is utilized for sensing a magnetic vector from an external location of the capsule endoscope.

4. The capsule endoscope of claim 1, wherein the processing unit has a built-in timer, and the processing unit acquires time of capturing the images according to time recorded by the timer when the image capturing units capture the images.

5. The capsule endoscope of claim 1, further comprising a timer, wherein the processing unit acquires time of capturing the images according to time recorded by the timer when the image capturing units capture the images.

6. The capsule endoscope of claim 1, further comprising a memory unit for storing the images captured by the image capturing units and the angles of capturing the images with the image capturing units.

7. The capsule endoscope of claim 1, further comprising a transmitting unit for transmitting the images and the angles of capturing the images with the image capturing units to a receiving unit in an external location of the capsule endoscope.

8. A capsule endoscope, comprising:
a shell having a containing space;
a plurality of image capturing units disposed in the containing space and arranged such that a capturing range of each of the image capturing units overlaps with a capturing range of at least one adjacent image capture unit;
a positioning unit disposed in the containing space for positioning each of the image capturing units; and
a processing unit disposed in the containing space for acquiring angles of capturing images with the image capturing units according to positioning results of the image capturing units with the positioning unit.

9. The capsule endoscope of claim 8, wherein the positioning unit positions each of the image capturing units by utilizing two vectors.

10. The capsule endoscope of claim 8, wherein the positioning unit comprises two sensing units, one of the sensing units is utilized for sensing a gravity vector, and the other one of the sensing units is utilized for sensing a magnetic vector from an external location of the capsule endoscope.
